(19) United States
(12) Patent Application Publication
(10) Pub. No.: US 2013/0237734 A1
(43) Pub. Date: Sep. 12, 2013

(54) Aromatization Catalyst and Methods of Preparing Same

(71) Applicant: An-Hsiang Wu, Kingwood, TX (US)

(72) Inventor: An-Hsiang Wu, Kingwood, TX (US)

(73) Assignee: CHEVRON PHILLIPS CHEMICAL COMPANY LP, The Woodlands, TX (US)

(21) Appl. No.: 13/413,930

(22) Filed: Mar. 7, 2012

(51) Int. Cl. C07C 7/00; B01J 37/08; B01J 29/22; B01J 37/00; B01J 29/068; B01J 29/74

(52) U.S. Cl. 585/418; 502/62

(57) ABSTRACT

A method comprising contacting a crystalline aluminosilicate with an organic acid to form an acid-treated support; contacting the acid-treated support with a Group IB metal compound and a Group IIIA element compound to form a catalyst precursor; and contacting the catalyst precursor with a silylating agent to form a silylated catalyst.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### TECHNICAL FIELD

The present disclosure relates generally to catalysts and catalyst systems. Specifically, the disclosure relates to methods of preparing an aromatization catalyst.

### BACKGROUND

The catalytic conversion of hydrocarbons into aromatic compounds, referred to as aromatization is an important industrial process. The aromatization reactions may include the dehydrogenation, isomerization, and/or hydrocracking of hydrocarbons, each of which generates aromatic products. These reactions are generally conducted in one or more aromatization reactors containing aromatization catalysts. Given their commercial importance, an ongoing need exists for improved aromatization catalysts and methods of preparing same.

### SUMMARY

Disclosed herein is a method comprising contacting a crystalline aluminosilicate with an organic acid to form an acid-treated support; contacting the acid-treated support with a Group IB metal compound and a Group IIIA element compound to form a catalyst precursor; and contacting the catalyst precursor with a silylating agent to form a silylated catalyst.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

Disclosed herein are methods of preparing aromatization catalysts comprising treating a support comprising a crystalline aluminosilicate with an organic acid to form an acid-treated support, contacting the acid-treated support with one or more promoters to form a catalyst precursor, contacting the catalyst precursor with a silylating agent to form a silyated catalyst, and calcining the silylated catalyst to produce an aromatization catalyst. The aromatization catalyst may convert hydrocarbons into products comprising aromatic compounds. In an embodiment, the aromatization catalyst described herein may display an improved performance, for example a higher conversion, yield, purity, etc. of user-desired aromatic compounds, when compared to an otherwise similar aromatization catalyst lacking the organic acid treatment. Hereinafter, the aromatization catalyst is referred to as a high performance acid-treated catalyst (HPAC) and the silylated catalyst is referred to as an HPAC precursor. Methods of preparing an HPAC precursor and an HPAC will be described in more detail later herein.

In an embodiment, a method of preparing an HPAC may initiate by contacting a support with an organic acid to form an acid-treated support.

In an embodiment, the support comprises a crystalline aluminosilicate. Crystalline aluminosilicates may include bound medium pore zeolites, large pore zeolites, or mixtures thereof. Examples of large pore zeolites suitable for use in this disclosure include, but are not limited to, L-zeolite, Y-zeolite, mordenite, omega zeolite, beta zeolite, and the like.

In an embodiment, the support comprises a zeolite. The term “zeolite” generally refers to a particular group of hydrated, crystalline metal aluminosilicates. These zeolites exhibit a network of SiO₄ and AlO₄ tetrahedra in which aluminum and silicon atoms are crosslinked in a three-dimensional framework by sharing oxygen atoms. In the framework, the ratio of oxygen atoms to the total of aluminum and silicon atoms may be equal to 2. The framework exhibits a negative electrovalence that typically is balanced by the inclusion of cations within the crystal such as metals, alkali metals, alkaline earth metals, or hydrogen. Examples of suitable zeolite frameworks include without limitation MFI, FAU, MAZ, MOR, LTL, PAR, OFF, STI, MTW, EPI, TON, MEL, FER, or combinations thereof. In an embodiment, the zeolite may have a pore size of from about 3 Angstrom (Å) to about 10 Å, alternatively from about 5 Å to about 8 Å.

In an embodiment, the support comprises a ZSM zeolite which has an MFI framework. Generally, the ZSM zeolite has a high silicon to aluminum ratio. For example, the ratio of SiO₂ to Al₂O₃ in the ZSM zeolite may be equal to or greater than about 5:1, alternatively from about 8:1 to about 200:1, alternatively from about 12:1 to about 100:1. Examples of suitable ZSM zeolites include without limitation ZSM-5, ZSM-8, ZSM-11, ZSM-12, ZSM-35, ZSM-38, or combinations thereof.

In an embodiment, the support comprises a ZSM-5, alternatively a protonated ZSM-5 (HZSM-5). ZSM-5 is composed of several pentasil units linked together by oxygen bridges to form pentasil chains. Typical ZSM-5 zeolites contain mole ratios of oxides in accordance with the following formula:

NaₙALₙSi₉₆₋ₙO₁₉₂.16H₂O

wherein n is from about 0 to about 27.

In an embodiment, the support may be precalcined prior to treatment with the organic acid. For example, the support may be precalcined at a temperature of from about 150° C. to about 600° C., alternatively from about 250° C. to about 575° C., alternatively from about 350° C. to about 550° C. The precalcination of the support may be carried out for a time period of from about 0.5 hour to about 16 hours, alternatively from about 0.5 hour to 8 hours, alternatively from about 1 hour to about 4 hours.

The support may be present in the HPAC in an amount of from about 5 weight percent (wt. %) to about 95 wt. % by total weight of the HPAC, alternatively from about 10 wt. % to about 90 wt. %, alternatively from about 20 wt. % to about 80 wt. %. Herein, weight percentage by total weight of the catalyst refers to the weight percentage of the component based on the final weight of the catalyst (i.e., HPAC) after all of the catalyst processing steps.

The support may further comprise a binder. In an embodiment the binder for use with the zeolite comprises synthetic or naturally occurring zeolites; alumina; clays such as montmorillonite and kaolin; the refractory oxides of metals of Groups IVA and IVB of the Periodic Table of the Elements; oxides of silicon, titanium, zirconium; or combinations thereof. In an embodiment, the binder comprises silica. In an embodiment, the silica particles may be in the form of a silica sol. A silica sol may be obtained by dispersing the silica particles in water. The silica sol may be provided in about 20 wt. % to about 30 wt. % aqueous solution having a pH of from about 9.0 to about 10.5 with a viscosity of equal to or less than about 20 mPa·s at 25° C., alternatively from about 1 mPa·s to about 20 mPa·s at 25° C.

The zeolite and binder (e.g., silica) may be combined in a weight ratio of from about 95:5 to about 50:50 zeolite:binder; alternatively from about 90:10 to about 70:30 zeolite:binder; alternatively from about 88:12 to about 78:22 zeolite:binder. The amount of water necessary to form an extrudable paste may be determined by one of ordinary skill in the art. In an embodiment, the amount of water necessary to form an extrudable paste comprising zeolites having a mean and median particle size within the disclosed ranges is reduced in comparison to an otherwise identical catalyst comprising zeolites having a mean and median particle size outside the disclosed ranges.

The amount of water may be sufficient to form a paste having a dough-like consistency. Such a paste may be characterized by a resistance to crumbling (e.g., not brittle) and the ability to maintain a cohesive form (e.g., not a soup-like consistency). The paste may be further characterized by an ability to form a plug at the die interface, which can then be expelled out through die openings in a cylindrical shape form resembling spaghetti strands.

In an embodiment, the paste is formed into shaped particles. In an embodiment, the paste may be formed into any suitable shape. Methods for shaping the paste are well known in the art, and include, for example, extrusion, spray drying, pelletizing, agglomeration and the like. In an embodiment, the paste is formed into an extrudate, for example as described in U.S. Pat. Nos. 5,558,851 and 5,514,362 each of which are incorporated herein in their entirety.

The organic acid may include any decomposable organic acid. Examples of organic acids suitable for use in this disclosure include without limitation formic acid, acetic acid, propionic acid, butyric acid, isobutyric acid, trimethylacetic acid, isovaleric acid, octanoic acid, oxalic acid, malonic acid, methylmalonic acid, ethylmalonic acid, butylmalonic acid, dimethylmalonic acid, succinic acid, methylsuccinic acid, dimethylsuccinic acid, glutaric acid, adipic acid, methyladipic acid, tertbutyladipic acid, sebacic acid, benzoic acid, phthalic acid, isophthalic acid, terephthalic acid, diphenic acid, naphthalaldehydic acid, methanesulfonic acid, p-toluenesulfonic acid, trichloroacetic acid, trifluoroacetic acid, or combinations thereof.

In an embodiment, the organic acid comprises at least two carboxylic acid (—COOH) groups. Such acids may function as multidentate ligands or chelating agents. In an embodiment, the organic acid comprises oxalic acid, ethylene diamine tetraacetic acid (EDTA), nitrilotriacetic acid, or combinations thereof. In an embodiment, the organic acid comprises oxalic acid.

The organic acid may be dissolved in water to form an aqueous solution. In such an embodiment, the organic acid may have a concentration of from about 0.2% weight/weight (w/w) to about 10% w/w, alternatively from about 0.5% w/w to about 5% w/w, alternatively from about 1% w/w to about 3% w/w.

In an embodiment, the support may be contacted with the organic acid in a weight ratio of organic acid:support of equal to or less than about 9:1, alternatively equal to or less than about 6:1, alternatively equal to or less than about 3:1 wherein the weight ratio is of dry organic acid to dry support.

The contacting of the support and organic acid may be carried out using any suitable method. For example, the support may be contacted with the organic acid by soaking the support in the organic acid at a temperature of equal to or less than about 100° C., alternatively from about 50° C. to about 100° C., alternatively from about 90° C. to about 100° C.; for a time period of from about 0.1 hour to about 100 hours, alternatively from about 0.5 hour to about 50 hours, alternatively from about 1 hour to about 24 hours. The resulting acid-treated support may be washed for example with water at room temperature for a time period of from about 1 minute to about 1 hour and then dried. The drying conditions may be at a temperature of from about 0 to about 200° C., alternatively from about 20° C. to about 150° C., alternatively at room temperature; and for a time period of from about 0.1 hours to about 100 hours, alternatively from about 0.5 hour to about 50 hours, alternatively from about 1 hour to about 24 hours. Herein room temperature refers to a temperature of from about 20° C. to about 26° C.

In an embodiment, the method further comprises contacting the acid-treated support with the one or more promoters to form a catalyst precursor. Herein, a promoter refers to a compound or composition which may function to enhance olefin production and/or suppress coke formation in an aromatization process. Examples of suitable promoters include without limitation metals from the Group IA, IIA, IIIA, IVA, VA, IIB, IIIB, IVB, VIB of the periodic table, or combinations thereof. Groups of elements of the table are indicated using the numbering scheme of the CAS version of the periodic table. In an embodiment, the promoter comprises silver (Ag). Silver may be added to the HPAC by contacting the acid-treated support with a silver-containing compound. The silver-containing compound may include any chemical that decomposes to produce silver in the support. Examples of suitable silver-containing compounds include without limitation silver acetate, silver carbonate, silver cyclohexanebutyrate, silver ethylhexanoate, silver nitrate, silver tetrafluoroborate, silver trifluoroacetate, or combinations thereof. In an embodiment, the silver-containing compound comprises silver nitrate (AgNO₃).

In an embodiment, the promoter comprises boron (B). Boron may be added to the HPAC by contacting the acid-treated support with a boron-containing compound. The boron-containing compound may include any chemical that decomposes to produce boron in the support. Examples of suitable boron-containing compounds include without limitation boric acid, carborane, phenylboronic acid, sodium tetrafluoroborate, tris(trimethylsiloxy)boron, triethanolamine borate, trialkyl borate, or combinations thereof. In an embodiment, the boron-containing compound comprises boric acid.

Contacting of the acid-treated support with one or more promoters may be carried out using any suitable method, such as for example incipient wetness impregnation. In an embodiment, the acid-treated support is impregnated with silver by contacting the acid-treated support with a silver-containing compound of the type described herein. In another embodiment, the acid-treated support is impregnated with boron by contacting the acid-treated support with a boron-containing compound of the type described herein. In yet another embodiment, the acid-treated support is contacted with both a silver-containing compound and a boron-containing compound of the types disclosed herein. Contacting the acid-treated support with the silver-containing compound and the boron-containing compound may be carried out simultaneously, alternatively the contacting may be carried out sequentially. In an embodiment, the acid-treated support is first contacted with a silver-containing compound, followed by a boron-containing compound to form a catalyst precursor.

In an embodiment, the catalyst precursor comprises silver in an amount of from about 0.5 wt. % to about 3.5 wt. % based on the total weight of the catalyst precursor, alternatively from about 1 wt. % to about 2.5 wt. %, alternatively from about 1.5 wt. % to about 2 wt. %. In another embodiment, the catalyst precursor comprises boron in an amount of from about 0.1 wt. % to about 2 wt. % based on the total weight of the catalyst precursor, alternatively from about 0.3 wt. % to about 0.7 wt. %, alternatively from about 0.4 wt. % to about 0.6 wt. %. In yet another embodiment, the catalyst precursor may have an atomic ratio of silver:boron of from about 1:1 to about 4.5:1, alternatively from about 2:1 to about 3.5:1, alternatively from about 2.5:1 to about 3:1.

The resulting catalyst precursor may be further processed by heating at a temperature of equal to or less than about 100° C., alternatively from about 50° C. to about 100° C., alternatively from about 90° C. to about 100° C.; for a time period of from about 1 hour to about 40 hours, alternatively from about 5 hours to about 30 hours, or alternatively from about 10 hours to about 20 hours. The temperature may then be reduced and maintained at a range of from about 15° C. to about 50° C., alternatively from about 20° C. to about 30° C. for a time period of from about 0 hour to 32 hours, or alternatively from about 0.1 hour to about 16 hours.

The catalyst precursor may be further calcined at a temperature of from about 150° C. to about 600° C., alternatively from about 250° C. to about 600° C., alternatively from about 350° C. to about 550° C. for a time period of from about 1 hour to about 24 hours, alternatively from about 2 hours to about 10 hours, alternatively from about 4 hours to about 8 hours.

In an embodiment, the method may further comprise contacting the catalyst precursor with a silylating agent to form an HPAC precursor. Herein, the silylating agent refers to silicon-containing materials which may function to reduce the rate of coke formation in the conversion of hydrocarbon to aromatic compounds. In an embodiment, the silylating agent comprises a silicon-containing compound having a general chemical formula R₁R₂R₃Si[OₘSiR₄R₅]ₙR₆ wherein R₁, R₂, R₃, R₄, R₅, and R₆ are each independently hydrogen, alkyl radical, alkenyl radical, alkoxy radical, aryl radical, aryloxy radical, alkaryl radical, aralkyl radical, or combinations thereof; m is 0 or 1; and n is from about 1 to about 10. For the purposes of this application, the term “alkyl(s)” or “alkyl radical(s)” refers to a univalent group derived by removal of a hydrogen atom from any carbon atom of an alkane. For the purposes of this application, the term “alkenyl(s)” or “alkenyl radical(s)” refers to an unsaturated chemical compound containing at least one carbon to carbon double bond. For the purposes of this application, the term “alkoxy(s)” or “alkoxy radical(s)” refers to a compound comprising an alkyl group linked to oxygen. For the purposes of this application, the term “aryl(s)” or “aryl radical(s)” refers to a functional group or substituent derived from a simple aromatic ring. For the purposes of this application, the term “aryloxy(s)” or “aryloxy radical(s)” refers to univalent radicals of the type Ar—O— where Ar is an aryl group. For the purposes of this application, the term “alkaryl(s)” or “alkaryl radical(s)” refers to radicals comprising alkylene-aryl groups having from 1 to 10 carbon atoms in the alkylene moiety and from 6 to 10 carbon atoms in the aryl moiety. For the purposes of this application, the term “aralkyl(s)” or “aralkyl radical(s)” refers to radical in which an aryl group is substituted for an alkyl hydrogen atom.

The silicon-containing compound may have a silicon concentration of from about 0.1 wt. % to about 80 wt. %, alternatively from about 1 wt. % to about 40 wt. %, alternatively from about 5 wt. % to about 20 wt. % based on added weight of silicon/total weight of catalyst. Examples of suitable silicon-containing compounds include without limitation silicon-containing polymer, silicon-containing oligomer, organosilicate, silane, or combinations thereof. Examples of silicon-containing polymers include without limitation poly(phenylmethylsiloxane), poly(phenylethylsiloxane), poly(phenylpropylsiloxane), hexamethyldisiloxane, decamethyltetrasiloxane, diphenyltetramethyldisiloxane, or combinations thereof. An example of an organosilicate includes without limitation tetraethyl orthosilicate. Examples of silanes include without limitation trimethylchlorosilane, chloromethyldimethylchlorosilane, N-trimethylsilylimidazole, N,O-bis(trimethylsilyl)acetimide, N-methyl-N-trimethylsilyltrifluoroacetamide, t-butyldimethylsilylimidazole, N-trimethylsilylacetamide, methyltrimethoxysilane, vinyltriethoxysilane, ethyltrimethoxysilane, propyltrimethoxysilane, (3,3,3-trifluoropropyl)trimethoxysilane, 3-(2-aminoethyl)aminopropyl]trimethoxysilane, cyanoethyltrimethoxysilane, aminopropyltriethoxysilane, phenyltrimethoxysilane, (3-chloropropyl)trimethoxysilane, (3-mercaptopropyl)trimethoxysilane, (3-glycidoxypropyl)trimethoxysilane, vinyltris(beta.-methoxyethoxy)silane, (gamma.-methacryloxypropyl)trimethoxysilane, vinylbenzyl cationic silane, (4-aminopropyl)triethoxysilane, gamma.-(beta-aminoethylamino)propyl]trimethoxysilane, (gamma-glycidoxypropyl)trimethoxysilane, (beta-(3,4-epoxycyclohexyl)ethyl)trimethoxysilane, (beta-mercaptoethyl)trimethoxysilane, (gamma-chloropropyl)trimethoxysilane, or combinations thereof. In an embodiment, the silicon-containing compound comprises tetraethyl orthosilicate. In another embodiment, the silicon-containing compound comprises poly(phenylmethyl)siloxane.

The silicon-containing compound may further comprise a diluent. The diluent may include any solubilizing fluid compatible with the other components of the HPAC. Examples of such fluids include without limitation hydrocarbons (e.g alkanes, cycloalkanes), alcohols, aromatics or mixtures thereof. In an embodiment, the diluent comprises cyclohexane. The silylating agent may be dissolved in a diluent such as a cyclohexane to form a silylating agent solution, which may be used to impregnate the catalyst precursor and form an HPAC precursor. In an embodiment, the silylating agent and diluent may form a solution having a silylating agent concentration of from about 0.1% w/w to about 80% w/w, alternatively from about 1% w/w to about 50% w/w, alternatively from about 2% w/w to about 30% w/w.

In an embodiment, the HPAC precursor may comprise a silylating agent in an amount of from about 0.1 wt. % to about 8 wt. % based on the total weight of the HPAC precursor, alternatively from about 0.5 wt. % to about 4 wt. %, alternatively from about 0.8 wt. % to about 2.5 wt. % based on weight of silicon added/total wt. These amounts may provide for a ratio of support:silylating agent of from about 99 to 1, alternatively from about 98 to 2, alternatively from about 80 to 20.

In an embodiment, the HPAC precursor comprises a support (e.g., HZSM-5) in an amount of from about 99 wt. % to about 90 wt. %, an organic acid (e.g. oxalic acid) in an amount of from about 0.1 wt. % to about 10 wt. %, silver in an amount of from about 0.1 wt. % to about 10 wt. %, boron in an amount of from about 0.1 wt. % to about 10 wt. %, and a silylating agent (e.g., poly(phenylmethyl)siloxane) in an amount of from about 0.1 wt. % to about 8 wt. % wherein the weight percentage is based on the total weight of the HPAC precursor.

The HPAC precursor may then be dried using drying conditions as described herein previously. The dried HPAC precursor may be calcined at a temperature of from about 150° C. to about 1000° C., alternatively from about 250° C. to about 750° C., alternatively from about 350° C. to about 650° C.; for a time period of from about 1 hour to 24 hours, alternatively from about 2 hours to about 10 hours, alternatively from about 4 hours to about 8 hours. The HPAC precursor having been treated as described previously herein is hereinafter referred to as the HPAC which may be employed as a catalyst in an aromatization reaction.

As will be understood by one of ordinary skill in the art, in some embodiments processing of the HPAC precursor (e.g. drying, calcining) to form the HPAC may result in a reduction or loss of some components used to prepare the HPAC precursor. For example, during calcining the organic acid may evaporate from the HPAC precursor. Consequently, the final catalyst composition (i.e., HPAC) may differ from that of the HPAC precursor. In an embodiment, the final catalyst composition after all processing steps (i.e., HPAC) comprises a support (e.g. HZSM-5) in an amount of from about 80 wt. % to about 99.9 wt. %, silver in an amount of from about 10 wt. % to about 0.1 wt. %, boron in an amount of from about 10 wt. % to about 0.1 wt. %, and a silylating agent (e.g., poly(phenylmethyl)siloxane) in an amount of from about 0.1 wt. % to about 8 wt. % wherein the weight percentage is based on the total weight of the catalyst.

In an embodiment, the HPAC prepared as disclosed herein is used as a catalyst in an aromatization reactor system comprising at least one aromatization reactor and its corresponding processing equipment. As used herein, the terms “aromatization,” “aromatizing” and “reforming” refer to the treatment of a hydrocarbon feed to provide an aromatics enriched product, which in one embodiment is a product whose aromatics content is greater than that of the feed. Typically, one or more components of the feed undergo one or more reforming reactions to produce aromatics. Some of the hydrocarbon reactions that occur during the aromatization operation include the dehydrogenation of cyclohexanes to aromatics, dehydroisomerization of alkylcyclopentanes to aromatics, dehydrocyclization of acyclic hydrocarbons to aromatics, or combinations thereof. A number of other reactions also occur, including the dealkylation of alkylbenzenes, isomerization of paraffins, hydrocracking reactions that produce light gaseous hydrocarbons, e.g., methane, ethane, propane and butane, or combinations thereof.

The aromatization reaction occurs under process conditions that favor the dehydrocyclization reaction and limit undesirable hydrocracking reactions. The pressures may be from about 0 pound per square inch gauge (psig) to about 500 psig, alternatively from about 25 psig to about 300 psig. The molar ratio of hydrogen to hydrocarbons may be from about 0.1:1 to about 20:1, alternatively from about 1:1 to about 6:1. The operating temperatures include reactor inlet temperatures from about 700° F. (371.1° C.) to about 1050° F. (565.5° C.), alternatively from about 900° F. (482.2° C.) to about 1000° F. (537.7° C.). Finally, the liquid hourly space velocity for the hydrocarbon feed over the aromatization catalyst may be from about 0.1 to about 10, alternatively from about 0.5 to about 2.5.

The composition of the feed is a consideration when designing catalytic aromatization systems. In an embodiment, the hydrocarbon feed comprises non-aromatic hydrocarbons containing at least six carbon atoms. The feed to the aromatization system is a mixture of hydrocarbons comprising C₆ to C₈ hydrocarbons containing up to about 10 wt. % and alternatively up to about 15 wt. % of C₅ and lighter hydrocarbons (C₅⁻) and containing up to about 10 wt. % of C₉ and heavier hydrocarbons (C₉⁺). Such low levels of C₉+ and C₅⁻ hydrocarbons maximize the yield of high value aromatics. In some embodiments, an optimal hydrocarbon feed maximizes the percentage of C₆ hydrocarbons. Such a feed can be achieved by separating a hydrocarbon feedstock such as a full range naphtha into a light hydrocarbon feed fraction and a heavy hydrocarbon feed fraction, and using the light fraction.

In another embodiment, the feed is a naphtha feed. The naphtha feed may be a light hydrocarbon, with a boiling range of about 70° F. (21.1° C.) to about 450° F. (232.2° C.). The naphtha feed may contain aliphatic, naphthenic, or paraffinic hydrocarbons. These aliphatic and naphthenic hydrocarbons are converted, at least in part, to aromatics in the aromatization reactor system. While catalytic aromatization typically refers to the conversion of naphtha, other feedstocks can be treated as well to provide an aromatics enriched product. Therefore, while the conversion of naphtha is one embodiment, the present disclosure can be useful for activating catalysts for the conversion or aromatization of a variety of feedstocks such as paraffinic hydrocarbons, olefinic hydrocarbons, acetylenic hydrocarbons, cyclic paraffin hydrocarbons, cyclic olefin hydrocarbons, and mixtures thereof, and particularly saturated hydrocarbons.

In an embodiment, the feedstock is substantially free of sulfur, nitrogen, metals, and other known poisons for aromatization catalysts. In an embodiment, the feedstock contains less than about 100 ppb of sulfur. If present, such poisons can be removed using any suitable methods. For example, the feed can be purified by first using conventional hydrofining techniques, then using sorbents to remove the remaining poisons.

The methodologies for preparation of an aromatization catalyst (i.e., HPAC) of the type disclosed herein may provide a catalyst with having higher performance when compared to an otherwise similar catalyst composition lacking the organic acid treatment as described herein. Hereinafter an otherwise similar catalyst composition lacking the organic acid treatment will be referred to as a base catalyst composition (BCC). For example, the HPAC may display an increased conversion of a mix C₅ hydrocarbons feed into a product comprising high value light olefins and benzene, toluene, ethylbenzene, xylene (BTEX) when compared to the BCC. The conversion of mix C₅ hydrocarbon may be determined by any suitable method such as for example analysis of the feed and product by gas chromatography. In an embodiment, an HPAC of the type described herein may be used in an aromatization process to produce a product having a mix C₅ conversion of from about 1% to about 100%, alternatively from about 2% to about 95%, alternatively from about 3% to about 90%.

In an embodiment, an HPAC of the type described herein may be used in an aromatization process to produce an increased BTEX yield when compared to a BCC. BTEX yield may be determined using any suitable methodology such as for example analysis of the product by gas chromatography. In an embodiment, the HPAC of the type described herein may be used in an aromatization process to produce a product having a BTEX yield of from about 0 to about 100%, alternatively from about 1% to about 90%, alternatively from about 2% to about 80%.

In an embodiment, an HPAC of the type described herein may be used in an aromatization process to produce a product having an increased BTEX purity when compared to the BCC. BTEX purity may be determined by total wt. % BTEX/total wt. % of C₆-C₈ as determined by gas chromatography. In an embodiment, a HPAC of the type described herein may be used in an aromatization process to produce a product having a BTEX purity of from about 0% to about 100%, alternatively from about 10% to about 99%, alternatively from about 20% to about 90%.

In some embodiments, the HPAC is prepared so as to retain the organic acid in the composition after all processing steps. In such embodiments, organic acid may improve the catalytic performance associated with the catalyst. The organic acid may disassociate from the catalyst over some time period as a function of the reaction conditions (e.g., temperature pressure). The catalyst may then exhibit a performance comparable to a BCC.

In an embodiment, the HPAC is amenable to regeneration by oxidative decoking. In an embodiment, the regeneration is a continuous process. Such as processes are disclosed for example in U.S. Pat. Nos. 6,124,515; 6,436,863; 6,420,295; and 6,235,954 each of which is incorporated by reference herein in its entirety.

### EXAMPLES

The invention having been generally described, the following examples are given as particular embodiments of the invention and to demonstrate the practice and advantages thereof. It is understood that the examples are given by way of illustration and are not intended to limit the specification of the claims to follow in any manner.

### Example 1

The performance of an HPAC was compared to two aromatization catalysts lacking the organic acid (e.g., oxalic acid) treatment. A control catalyst, designated Catalyst 1, was Zeolite T-4480, which is a protonated ZSM-5 (HZSM-5) catalyst commercially available from United Catalysts Inc. Catalyst 1 was precalcined at 538° C. for 2 hours. Catalyst 2 was prepared by adding a solution of 1.76 wt. % silver nitrate dissolved in water and a solution of 0.48 wt. % of boric acid dissolved in water to a precalcined Zeolite T-440 to form a mixture. The mixture was then processed by heating at 95° C. for 16 hours. Subsequently, the temperature was lowered to room temperature, where the mixture was held for 8 hours. Next, the mixture was calcined at 538° C. for 6 hours. A 10 wt. % solution of poly(methylphenylsilane) dissolved in cyclohexane was added to the mixture before calcining the composition again at 538° C. for 6 hours.

Catalyst 3 was prepared by adding large excess of 2 wt. % oxalic acid to a sample of precalcined Zeolite T-4480 to form mixture 1. Mixture 1 was then heated at 95° C. for 2 hours, the solution was decanted, washed with water, dried at room temperature and then calcined at 538° C. for 6 hours. Next, a solution of 1.68 wt. % of silver nitrate dissolved in water and a solution of 0.46 wt. % boric acid dissolved in water were added to mixture 1. The mixture was then processed as described for Catalyst 2.

Each catalyst was then evaluated for their ability to convert a gasoline cut of mix C5, BTEX yield and BTEX purity. A 1.0 g sample of each catalyst described above was placed into a stainless steel tube reactor (length: about 2 inches, inner diameter about 0.25 inches). A mixture of C5's from a catalytic cracking unit of a refinery was passed through the reactor at a flow rate of about 0.22 ml/min, a temperature of about 925° F., and an atmospheric pressure of 0 psig. The resulting liquid product exiting the reactor tube was analyzed (at hourly intervals) by gas chromatography. Table 1 presents the performance of each catalyst after 8 hours on stream.

TABLE 1

| | Pre- | Catalyst | Post- | ΣC5 Conversion | BTEX Yield | BTEX Purity |
|---|---|---|---|---|---|---|
| Catalyst | Treatment | Promoter | Treatment | (wt. %) | (wt. %) | (%) |
| 1 | n/a | n/a | n/a | 90.914 | 15.195 | 83.461 |
| 2 | n/a | Ag-B | PMPS | 76.984 | 12.329 | 73.470 |
| 3 | Oxalic Acid | Ag-B | PMPS | 99.863 | 44.323 | 97.270 |

The results demonstrated that Catalyst 3 which was treated with oxalic acid provided the highest C5 conversion, BTEX yield, and BTEX purity.

While embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R_{L}, and an upper limit, R_{U}, is disclosed, any number falling within the range is specifically disclosed. In particular, the following numbers within the range are specifically disclosed: R=R_{L}+k*(R_{U}−R_{L}), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference herein is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

What is claimed is:

1. A method comprising:
contacting a crystalline aluminosilicate with an organic acid to form an acid-treated support;
contacting the acid-treated support with a Group IB metal compound and a Group IIIA element compound to form a catalyst precursor; and
contacting the catalyst precursor with a silylating agent to form a silylated catalyst.

2. The method of claim 1 wherein the contacting with the organic acid comprises soaking the support with the organic acid at a temperature of up to about 100° C.

3. The method of claim 1 further comprising precalcining the support at a temperature of from about 150° C. to about 600° C. for a time period of from about 0.5 hour to about 16 hours prior to contacting with the organic acid.

4. The method of claim 1 further comprising:
heating the acid-treated support at a temperature of up to about 100° C. for a time period of from about 0.1 hour to about 100 hours to form a heated acid-treated support;
washing the heated acid-treated support to form a washed acid-treated support;
drying the washed acid-treated support at room temperature for a time period of from about 1 minute to about 24 hours to form a dried acid-treated support; and
calcining the dried acid-treated support at a temperature of from about 150° C. to about 600° C. for a time period of from about 1 hour to about 24 hours prior to contacting with the Group IB metal and the Group IIIA element.

5. The method of claim 1 wherein the contacting of the support with the organic acid is at a ratio of organic acid:support of equal to or less than about 9:1.

6. The method of claim 1 wherein the crystalline aluminosilicate comprises a bound zeolite.

7. The method of claim 6 wherein the zeolite has a framework of MFI, FAU, MAZ, MOR, LTL, PAR, OFF, STI, MTW, EPI, TON, MEL, FER, or combinations thereof.

8. The method of claim 6 wherein the zeolite has a framework of MFI.

9. The method of claim 1 wherein the organic acid comprises formic acid, acetic acid, propionic acid, butyric acid, isobutyric acid, trimethylacetic acid, valeric acid, isovaleric acid, hexanoic acid, octanoic acid, oxalic acid, malonic acid, methylmalonic acid, ethylmalonic acid, butylmalonic acid, dimethylmalonic acid; succinic acid, methylsuccinic acid, dimethylsuccinic acid, glutaric acid, adipic acid, methyladipic acid, tert butyladipic acid, sebacic acid, benzoic acid, phthalic acid, isophthalic acid, terephthalic acid, diphenic acid, naphthalaldehydic acid, methanesulfonic acid, p-toluenesulfonic acid, trichloroacetic acid, trifluoroacetic acid, or combinations thereof.

10. The method of claim 1 wherein the organic acid comprises oxalic acid, malonic acid, succinic acid, or combinations thereof.

11. The method of claim 1 wherein the organic acid is an aqueous solution having a concentration of from about 0.2 wt. % to about 10 wt. % based on the total weight of the aqueous solution.

12. The method of claim 1 further comprising:
heating the catalyst precursor at a temperature of up to 100° C. for a time period of from about 1 hour to about 40 hours;
holding the catalyst precursor at a temperature of from about 15° C. to about 50° C. for a time period of from about 0 hour to 32 hours; and
calcining the catalyst precursor at a temperature of from about 150° C. to about 600° C. for a time period of from about 1 hour to about 24 hours prior to contacting with the silylating agent.

13. The method of claim 1 wherein the Group IB metal compound comprises a silver-containing compound and the Group IIIA element compound comprises a boron-containing compound.

14. The method of claim 13 wherein the silver-containing compound comprises silver acetate, silver carbonate, silver cyclohexanebutyrate, silver ethylhexanoate, silver nitrate, silver tetrafluoroborate, silver trifluoroacetate, or combinations thereof.

15. The method of claim 13 wherein the boron-containing compound comprises boric acid, carborane, phenylboronic acid, sodium tetrafluoroborate, tris(trimethylsiloxy)boron, or combinations thereof.

16. The method of claim 1 wherein the catalyst precursor has a silver:boron molar ratio of from about 1:1 to about 4.5:1.

17. The method of claim 1 wherein the catalyst precursor comprises
a silver-containing compound in an amount of from about 0.5 wt. % to about 3.5 wt. % based on the total weight of the catalyst precursor; and
a boron-containing compound in an amount of from about 0.1 wt. % to about 2 wt. % based on the total weight of the catalyst precursor.

18. The method of claim 1 wherein the silylating agent has a chemical formula of R₁R₂R₃Si[OₘSiR₄R₅]ₙR₆,
wherein R₁, R₂, R₃, R₄, R₅, and R₆ are each independently hydrogen, alkyl radical, alkenyl radical, alkoxy radical, aryl radical, aryloxy radical, alkaryl radical, aralkyl radical, or combinations thereof;
m is 0 or 1; and
n is from 1 to 10.

19. The method of claim 1 wherein the silylating agent comprises silicon-containing polymer, silicon-containing oligomer, organosilicate, silane, or combinations thereof.

20. The method of claim 1 wherein the silylating agent comprises poly(phenylmethylsiloxane), poly(phenylethylsiloxane), poly(phenylpropylsiloxane), hexamethyldisiloxane, decamethyltetrasiloxane, diphenyltetramethyldisiloxane, or combinations thereof.

21. The method of claim 1 wherein the silylating agent comprises organosilicate, tetraethyl orthosilicate, or combinations thereof.

22. The method of claim 1 wherein the silylating agent comprises trimethylchlorosilane, chloromethyldimethylchlorosilane, N-trimethylsilylimidazole, N,O-bis(trimethylsilyl)acetimide, N-methyl-N-trimethylsilyltrifluoroacetamide, t-butyldimethylsilylimidazole, N-trimethylsilylacetamide, methyltrimethoxysilane, vinyltriethoxysilane, ethyltrimethoxysilane, propyltrimethoxysilane, (3,3,3-trifluoropropyl)trimethoxysilane, 3-(2-aminoethyl)aminopropyl]trimethoxysilane, cyanoethyltrimethoxysilane, aminopropyltriethoxysilane, phenyltrimethoxysilane, (3-chloropropyl)trimethoxysilane, (3-mercaptopropyl)trimethoxysilane, (3-glycidoxypropyl)trimethoxysilane, vinyltris(beta.-methoxyethoxy)silane, (gamma.-methacryloxypropyl)trimethoxysilane, vinylbenzyl cationic silane, (4-aminopropyl)triethoxysilane, gamma.-(beta-aminoethylamino)propyl]trimethoxysilane, (gamma-glycidoxypropyl)trimethoxysilane, (beta-(3,4-epoxycyclohexyl)ethyl)trimethoxysilane, (beta-mercaptoethyl)trimethoxysilane, (gamma-chloropropyl)trimethoxysilane, or combinations thereof.

23. The method of claim 1 wherein the silylating agent comprises poly(phenylmethylsiloxane), tetraethyl orthosilicate, or combinations thereof.

24. The method of claim 1 wherein the silylating agent has a concentration of from about 0.1 wt. % to about 80 wt. % based on the total weight of the diluted silylating agent.

25. The method of claim 1 wherein the silylated catalyst comprises:
a silver containing compound in an amount of from about 0.1 wt. % to about 10 wt. % based on the total weight of the silylated catalyst;
a boron containing compound in an amount of from about 0.1 wt. % to about 10 wt. % based on the total weight of the silylated catalyst; and
a silylating agent in an amount of from about 0.1 wt. % to about 8 wt. % based on the total weight of the silylated catalyst.

26. The method of claim 1 further comprising calcining the silylated catalyst at a temperature of from about 150° C. to about 1000° C. for a time period of from about 1 hour to about 24 hours to form an aromatization catalyst suitable for use in an aromatization process.

27. The method of claim 26 wherein the aromatization catalyst comprises:
a crystalline aluminosilicate in an amount of from about 80 wt. % to about 99 wt. % based on the total weight of the catalyst;
a silver-containing compound in an amount of from about 0.1 wt. % to about 10 wt. % based on the total weight of the catalyst;
a boron-containing compound in an amount of from about 0.1 wt. % to about 10 wt. % based on the total weight of the catalyst; and
a silylating agent in an amount of from about 0.1 wt. % to about 8 wt. % based on the total weight of the catalyst.

28. The method of claim 26 further comprising contacting the aromatization catalyst with a hydrocarbon feed in a reaction zone under suitable reaction conditions to form aromatic compounds and olefins and recovering a product comprising the aromatic compounds and olefins from the reaction zone.

29. The method of claim 28 wherein the product has a mix C5 conversion of from about 1% to about 100%.

30. The method of claim 28 wherein the product has a BTEX yield of from about 0% to about 100%.

31. The method of claim 28 wherein the product has a BTEX purity of from about 0% to about 100%.
